(19) United States
(12) Patent Application Publication
(10) Pub. No.: US 2014/0336453 A1
(43) Pub. Date: Nov. 13, 2014

(54) PRESSURE SENSOR, ENDOSCOPE AND ENDOSCOPE DEVICE

(71) Applicants: Masaru Ueki, Yonago-shi (JP); Kazutake Uehara, Tottori-shi (JP); Tsuyoshi Sasaki, Yonago-shi (JP)

(72) Inventors: Masaru Ueki, Yonago-shi (JP); Kazutake Uehara, Tottori-shi (JP); Tsuyoshi Sasaki, Yonago-shi (JP)

(73) Assignees: JAPAN MICRO SYSTEM CO., LTD., Yonago-shi (JP); NATIONAL UNIVERSITY CORPORATION TOTTORI UNIVERSITY, Tottori-shi (JP)

(21) Appl. No.: 14/116,308

(22) PCT Filed: May 7, 2012
(86) PCT No.: PCT/JP2012/061651

§ 371 Date: Jul. 3, 2014

(30) Foreign Application Priority Data
May 9, 2011 (JP) 2011-104389

(51) Int. Cl. A61B 1/00; A61B 1/04

(52) U.S. Cl. CPC A61B 1/00055; A61B 1/04; A61B 1/00045
USPC 600/103

(57) ABSTRACT

There are provided a pressure sensor which can avoid an endoscope from causing intestinal perforation, an endoscope including the pressure sensor, and an endoscope device including the endoscope. There is provided a pressure sensor including a support unit and a pressure sensitive unit disposed on the ridge line of the front end of the support unit and configured to output a signal corresponding to an applied pressure. The pressure sensitive unit includes a first electrode disposed in a ring shape and multiple second electrodes opposed to the first electrode. The second electrodes are spaced in the circumferential direction of the first electrode.

### TECHNICAL FIELD

The present invention relates to a pressure sensor which is suitably mounted on an endoscope, an endoscope including the pressure sensor, and an endoscope device including the endoscope.

### BACKGROUND ART

The number of deaths by cancer in Japan reached about 330,000 in 2005, about 13% of which is estimated to have been caused by colorectal cancer. It is said that colorectal cancer will take first place among the causes of death of the Japanese around 2015. While the most effective colorectal cancer treatment method is early detection and treatment by endoscopy, one problem thereof in clinical practice is that the doctor must be skilled in colorectal endoscopic procedures. Typically, the doctor determines the strength with which the endoscope is pushing the intestinal wall, based on endoscopic intestinal images, feelings transmitted to a hand of the doctor who is operating the endoscope, and a complaint of pain from the patient.

Known endoscope devices which can acquire intestinal information using methods other than images or feelings include an endoscope device described in Patent Document 1. In the endoscope device described in Patent Document 1, as shown in FIG. 34 and the like thereof, many capacitive sensors are disposed on a side surface adjacent to the front end of a probe. When the many sensors sense the pressure independently, the endoscope device acquires information about the degree of strength with which the probe is contacting the living body.

### PRIOR ART DOCUMENTS

### Patent Documents

[Patent Document 1] Japanese Unexamined Patent Application Publication No. 6-209902

### SUMMARY OF THE INVENTION

### Problems to be Solved by the Invention

The colorectal lumen has many ridges and grooves, as well as flexions. For this reason, the possibility that lesions behind the ridges and grooves may be overlooked and the risk that the endoscope may perforate an intestinal flexion have been pointed out. In particular, intestinal perforation has a high risk of becoming severe. Accordingly, it is an urgent task to develop an endoscope which can avoid intestinal perforation without depending on the capability of the operator.

The present invention has been made in view of the foregoing and provides a pressure sensor which can avoid endoscopic intestinal perforation, an endo scope including the pressure sensor, and an endoscope device including the endoscope.

### Means for Solving the Problems

The present invention provides a pressure sensor including a support unit and a pressure sensitive unit disposed on a ridge line of a front end of the support unit and configured to output a signal corresponding to an applied pressure. The pressure sensitive unit includes a first electrode disposed in a ring shape and multiple second electrodes opposed to the first electrode. The second electrodes are spaced in a circumferential direction of the first electrode.

The inventors have eagerly investigated the cause of endoscopic intestinal perforation. As a result, the inventors have acquired knowledge that intestinal perforation tends to occur when the operator moves the front end of the insertion unit of the endoscope and thus the ridge line of the front end strongly pushes the intestine. Based on this knowledge, the inventors have acquired the following knowledge: if it is possible to detect the magnitude and position of a pressure applied to the ridge line of the front end of the insertion unit, an excessive pressure applied to the ridge line can be detected before intestinal perforation occurs, and by operating the endoscope on the basis of the pressure detected, intestinal perforation can be prevented. The inventors have then found that if a pressure sensitive unit is formed on the ridge line of the front end of the insertion unit by disposing a first electrode in a ring shape and then spacing multiple second electrodes in the circumferential direction of the first electrode, it is possible to accurately measure the magnitude and position of a pressure applied to the ridge line. In this way, the inventors have reached the present invention.

The pressure sensitive unit outputs a signal corresponding to the applied pressure. For example, the pressure sensitive unit may be a pressure sensitive unit having a pressure sensitive resistor between the first and second electrodes and configured to detect a resistance variation therebetween, or may be a pressure sensitive unit having space between the first and second electrodes and configured to detect a capacitance variation therebetween. Since the resistance or capacitance between the first and second electrodes varies with the pressure applied to the ridge line, it is possible to detect the magnitude of the pressure applied to the ridge line.

Since the multiple second electrodes are disposed, the resistance variation or capacitance variation detected by each second electrode varies depending on the position to which the pressure is applied. For example, assuming that four second electrodes, A, B, C, and D, are disposed in the circumferential direction of the ring-shaped first electrode, when a pressure is applied to the vicinity of the second electrode A, the resistance variation or capacitance variation detected by the second electrode A becomes larger than those detected by the second electrodes B, C, and D. Thus, it is detected that the pressure has been applied to the vicinity of the second electrode A.

The magnitude and position of the pressure applied to the ridge line can be accurately measured on the above principle. While the functions and effects of the pressure sensor of the present invention have been described above using the case where the pressure sensor of the present invention is mounted on an endoscope, the pressure sensor of the present invention may be mounted not only on endoscopes but also on various types of apparatuses, including medical and industrial apparatuses. The pressure sensor can also be used to control various types of robots, since it is possible to determine the magnitude and position of the pressure received by the pressure sensor on a principle similar to that described above.

There have been developed self-propelled endoscopes in recent years, which autonomously travels the intestine using power generated by a motor, pneumatic pressure, or the like. However, such endoscopes do not allow the operator to feel a pressure applied to the front end of the insertion unit. Accordingly, even when runaway of the motor, or the like causes application of a strong pressure to the front end of the insertion unit and thus intestinal perforation may occur, the operator is less likely to become aware of this situation. By mounting the pressure sensor of the present invention on such an endoscope, it is possible to detect a pressure applied to the front end of the insertion unit and thus to prevent the self-propelled endoscope from causing intestinal perforation.

Intestinal perforation tends to occur when the front end of the endoscope is bent into a J-shape in order to observe the back of the ridges and grooves of the intestine. One method for preventing intestinal perforation from occurring for such a reason is to objectively display the applied pressure by mounting the pressure sensor on the front end of an endoscope. Another method is to increase the viewing angle of a lens disposed at the front end of an endoscope as much as possible. The viewing angle of the lens is, for example, 160, 180, 200, 220, 240, 260, 280, 300, 320, 340, or 360 degrees, or may be greater than or equal to one of the values presented or may be a value in a range between any two of the values.

The endoscope probe described in Patent Document 1 may be able to detect a pressure applied to a side surface of the insertion unit. However, it is very likely to be incapable of detecting a pressure applied to the front end of the insertion unit or to detect a pressure lower than the actually applied pressure. Accordingly, this endoscope probe is believed to have difficulty in preventing intestinal perforation effectively.

Hereafter, various embodiments of the present invention will be exemplified. Any of the embodiments described below can be combined with each other.

The pressure sensitive unit of the present invention preferably further includes a pressure sensitive resistor disposed between the first and second electrodes, a resistance of the pressure sensitive resistor varying with an applied pressure.

Preferably, the pressure sensitive resistor includes an elastomer including a conductive material.

Preferably, the pressure sensitive resistor surrounds the first or second electrode in a section perpendicular to a circumferential direction of the first or second electrode.

Preferably, the support unit has a ring-shaped recess on the ridge line, and at least one of the first and second electrodes, and the pressure sensitive resistor are disposed in the recess.

Preferably, a section of the pressure sensitive resistor is circular, the section being perpendicular to a circumferential direction of the ridge line, and a section of the recess is in an arc shape, the section being perpendicular to the circumferential direction of the ridge line.

Preferably, the pressure sensitive resistor extends off east one of a side and a front of the support unit.

Preferably, the second electrodes are disposed on the support unit, and the pressure sensitive resistor is fixed to the support unit using an adhesive or pressure sensitive adhesive disposed between adjacent two second electrodes.

Preferably, the second electrodes include four or more second electrodes uniformly spaced in the circumferential direction of the first electrode.

In another aspect, the present invention provides an endoscope including the pressure sensor described above and an insertion unit configured to be inserted into a body. The support unit constitutes a front end of the insertion unit.

In yet another aspect, the present invention provides an endoscope including the pressure sensor described above and an insertion unit configured to be inserted into a body. The support unit is an adapter configured to detachably fix the pressure sensitive unit to the insertion unit.

In still yet another aspect, the present invention provides an endoscope device including the endoscope described above, a signal processing unit configured to acquire a magnitude of a pressure applied to the pressure sensitive unit and a position to which the pressure has been applied, based on a signal from the pressure sensitive unit, and a monitor configured to display an image captured by the endoscope.

Preferably, the signal processing unit makes a display based on the magnitude of the applied pressure in a position which is located adjacent to the image and which corresponds to the pressure-applied position.

Preferably, the endoscope device further includes an external display unit which differs from the monitor, and the signal processing unit makes a display based on the magnitude of the applied pressure in a position on the external display unit, the position corresponding to the pressure-applied position.

Preferably, the display is a color corresponding to the magnitude of the applied pressure.

### BRIEF DESCRIPTION OF THE DRAWINGS

FIG. 1 shows an endoscope device 1 of one embodiment of the present invention.

FIG. 2(a) is a view of an insertion unit 9 seen from near a front end 13. FIG. 2(b) is a side view of the insertion unit 9. FIG. 2(c) is a sectional view taken along line A-A of FIG. 2(b). FIG. 2(d) is a sectional view taken along line B-B of FIG. 2(a).

FIG. 3 corresponds to FIG. 2(d) and shows another embodiment of a pressure sensitive unit 15.

FIG. 4 corresponds to FIG. 2(d) and shows yet another embodiment of the pressure sensitive unit 15.

FIG. 5 corresponds to FIG. 2(c) and shows another embodiment of a first electrode 25.

FIG. 6 corresponds to FIG. 2(a) and shows a pressure-applied portion P.

FIG. 7 corresponds to FIG. 2(a) and shows a pressure-applied portion P.

FIG. 8 is a diagram showing a method of making a display 31 based on the magnitude of an acquired applied pressure in a position which is located adjacent to an image 29 captured by the endoscope 3 and which corresponds to an acquired pressured-applied position.

FIG. 9 shows an external display unit 33 which differs from a monitor 7.

FIG. 10(a) is a view of an adapter 37 seen from near the front end thereof. FIG. 10(b) is a side view of the adapter 37. FIG. 10(c) is a sectional view taken along line A-A of FIG. 10(b). FIG. 10(d) is a sectional view taken along line B-B of FIG. 10(a).

FIGS. 11(a) to 11(d) show another embodiment of the pressure sensitive unit 15, in which FIG. 11(a) is a view of an insertion unit 9 seen from near a front end 13; FIG. 11(b) is a side view of the insertion unit 9; FIG. 11(c) is a sectional view taken along line A-A of FIG. 11(b); and FIG. 11(d) is a sectional view taken along line B-B of FIG. 11(a).

FIG. 12 shows an endoscope model used in an Example.

FIG. 13 shows pressure-applied positions of a pressure sensitive unit of the endoscope model of FIG. 12.

FIG. 14 shows voltages detected in the pressure-applied positions shown in FIG. 13.

FIG. 15 shows an example of pressure-resistance characteristics of an elastomer resistor which can be used in the present invention.

### EMBODIMENTS

Now, an embodiment of the present invention will be described with reference to the drawings. Note that the embodiment is illustrative only and does not limit the scope of the present invention.

1. Overall configuration of Endoscope Device

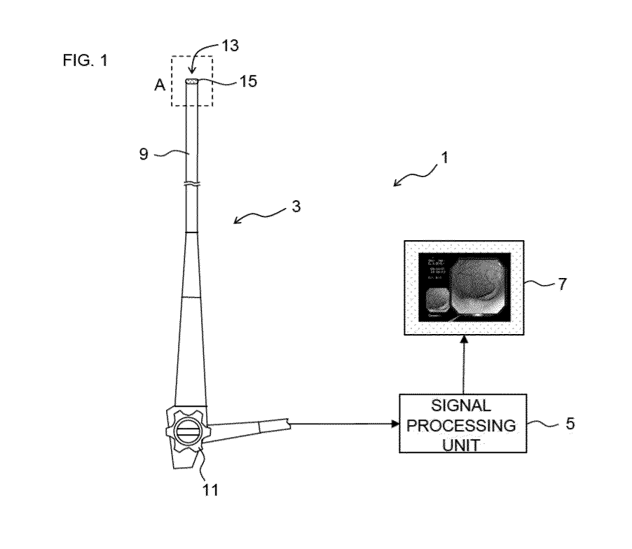

FIG. 1 shows an endoscope device 1 of one embodiment of the present invention. The endoscope device 1 of the present embodiment includes an endoscope 3, a signal processing unit 5, and a monitor 7 for displaying an image captured by the endoscope 3. The endoscope 3 includes an insertion unit 9 configured to be inserted into the body and an operation unit 11 used to perform an operation such as bending of a front end 13 of the insertion unit 9. The insertion unit 9 has a pressure sensitive unit 15 on the ridge line of the front end 13.

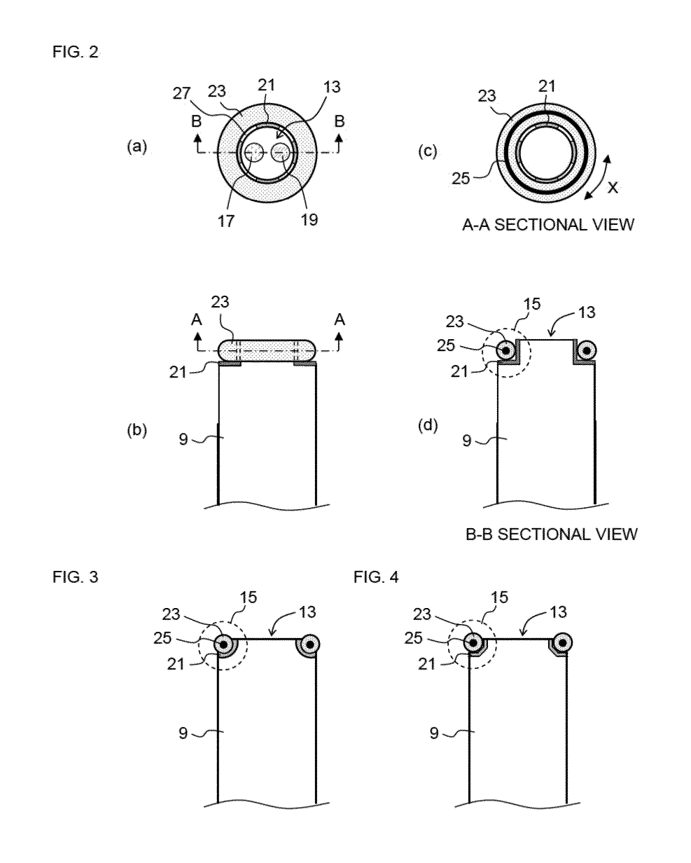

The front end 13 of the insertion unit 9 is provided with a projector 17 and an image capturing unit. 19 shown in FIG. 2(a) (to be discussed later). Light emitted by the projector 17 is reflected by the intestinal wall and then received by the image capturing unit 19. Thus, an intestinal image is captured. The configuration of the projector 17 is not limited to a particular one. For example, the projector 17 is configured as follows: light emitted by a light source passes through a light guide disposed in the insertion unit 9 and exits from the front end 13 of the insertion unit 9. The configuration of the image capturing unit 19 is not limited to a particular one. For example, light reflected by the intestinal wall is condensed by a objective lens disposed at the front end 13 and then received by an image sensor disposed at the image forming position of the objective lens, and an obtained image signal is transmitted to the signal processing unit 5 through a signal line disposed in the insertion unit 9, thereby displaying an intestinal image on the monitor 7.

For a traditional endoscope device, when the doctor operating the endoscope 3 inserts the insertion unit 9 into the intestine of the patient, he or she determines whether the front end 13 of the insertion unit 9 is contacting the intestinal wall, based on intestinal images being displayed on the monitor 7 and feelings transmitted to a hand of the doctor when the front end 13 of the insertion unit 9 contacts the intestinal wall. The doctor then inserts the insertion unit 9 into a deeper part of the body. If the doctor is experienced, he or she can accurately determine the strength with which the front end 13 is contacting the intestinal wall. When the front end 13 is strongly contacting the intestinal wall, the doctor operates the operation unit 11 to change the bending direction of the front end 13 of the insertion unit 9. Thus, he or she can avoid the front end 13 from applying an excessive pressure to the intestinal wall. If the doctor is less experienced, he or she may not well understand the relationship between feelings transmitted to the hand and the pressure applied to the intestinal wall by the front end 13. This may cause a problem that although the front end 13 is already strongly contacting the intestinal wall, he or she may continue to insert the insertion unit 9 without changing the bending direction of the front end 13 of the insertion unit 9 and thus cause intestinal perforation.

In the endoscope device 1 of the present embodiment, the pressure sensitive unit 15 disposed on the ridge line of the front end 13 of the insertion unit 9 detects a pressure being applied to the intestinal wall by the front end 13. When the pressure sensitive unit 15 detects that the front end 13 of the insertion unit 9 inserted into the intestine by the doctor is strongly contacting the intestinal wall, the doctor receives an alarm through the monitor 7 and then operates the operation unit 11 on the basis of the alarm to change the bending direction of the front end 13 of the insertion unit 9. Thus, he or she can prevent intestinal perforation. Traditionally, the supervisor, who is next to the operator and observing the situation, cannot objectively grasp feelings transmitted to the hand of the operator and thus cannot prevent an accident although he or she is next to the operator. On the other hand, by using the endoscope device 1 of the present embodiment, the supervisor can objectively grasp the situation from an alarm displayed on the monitor 7 and thus prevent an accident by taking a necessary measure when appropriate.

### 2. Configuration of Front End Portion of Insertion Unit

Hereafter, the configuration of a portion around the front end 13 of the insertion unit 9 (front end portion) will be described in more detail with reference to FIGS. 2(a) to 2(d) and FIGS. 3 to 7, which are enlarged views of an area A of FIG. 1.

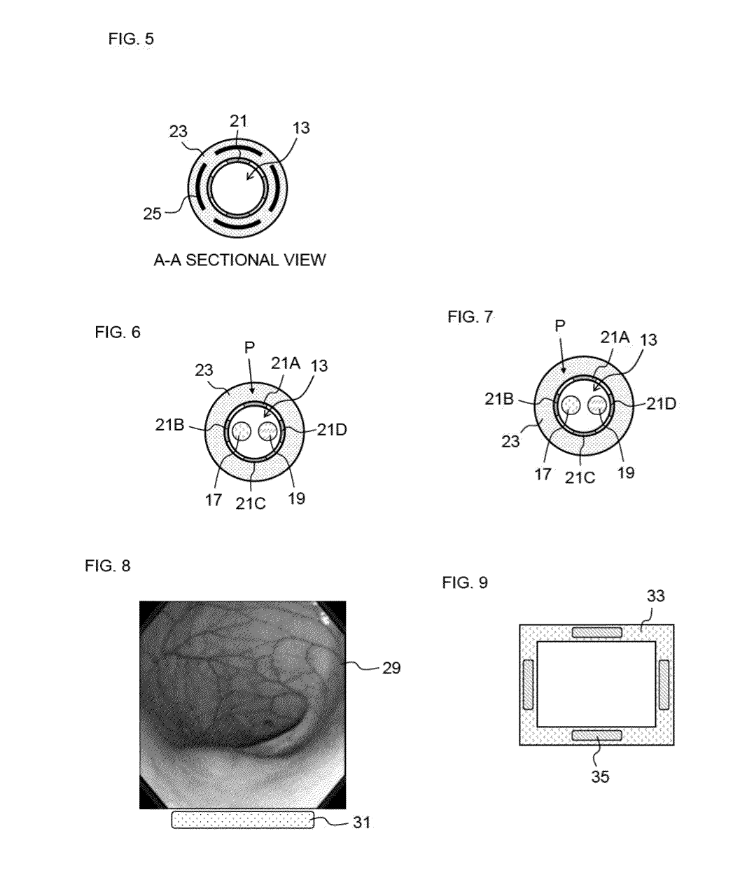

FIG. 2(a) is a view of the insertion unit 9 seen from near the front end 13. FIG. 2(b) is a side view of the insertion unit 9. FIG. 2(c) is a sectional view taken along line A-A of FIG. 2(b). FIG. 2(d) is a sectional view taken along line B-B of FIG. 2(a). FIG. 3 corresponds to FIG. 2(d) and shows another embodiment of the pressure sensitive unit 15. FIG. 4 corresponds to FIG. 2(d) and shows yet another embodiment of the pressure sensitive unit 15. FIG. 5 corresponds to FIG. 2(c) and shows another embodiment of a first electrode 25. FIGS. 6 and 7 correspond to FIG. 2(a) and show a pressure-applied portion P. The projector 17 and the image capturing unit 19 are omitted in the diagrams other than FIGS. 2(a), 6, and 7.

### 2-1. Pressure Sensitive Unit.

As shown in FIG. 2(d), the pressure sensitive unit 15 is disposed on the ridge line of the front end 13 of the insertion unit 9. The ridge line of the front end 13 refers to the perimeter of the front end 13. In other words, the ridge line is a portion where the side surface and front end 13 of the insertion unit 9 intersect each other. The pressure sensitive unit 15 includes the first electrode 25, second electrodes 21, and a pressure sensitive resistor 23 between these electrodes. The pressure sensitive unit. 15 of the present embodiment is simply configured and therefore has a low manufacturing cost advantage.

### 2-2. First Electrode

As shown in FIG. 2(c), the first electrode 25 is disposed in a ring shape along the perimeter of the front end 13. The description “disposed in a ring shape” includes a case where a single first electrode 25 having a closed ring shape is disposed along the perimeter of the front end 13 as shown in FIG. 2(c), as well as a case where multiple first electrodes 25 forming a ring shape as a whole are disposed as shown in FIG. 5. The term “ring” includes not only closed rings but also partially opened rings as shown in FIG. 5. The first electrode 25 is electrically connected to the signal processing unit 5 through a signal line disposed in the insertion unit 9 as shown in FIG. 2(c) or through a signal line or the like disposed along the outside of the insertion unit 9. When the number of first electrodes 25 is one, wiring from the first electrode 25 to the signal processing unit 5 is easier than that when the number is two or more.

### 2-3. Second Electrode

As shown in FIGS. 2(c) and 2(d), the second electrodes 21 are disclosed as opposed to the first electrode 25. The multiple second electrodes 21 are spaced in the circumferential direction of the first electrode (the direction of arrow X of FIG. 2(c)). Different signal lines are connected to the second electrodes 21, respectively, so as to measure the voltage between each second electrode 21 and the first electrode. This means that the number of pressure sensors corresponding to the number of second electrodes 21 are disposed. Accordingly, as the number of second electrodes 21 increases, the circumferential resolution of the pressure-applied position increases. The number of second electrodes 21 only has to be two or more and is preferably four or more, more preferably 8 or more. If the number of second electrodes 21 is four, the second electrodes 21 are disposed, for example, on the upper, lower, left, and right, sides of the section A-A, one on each side. If the number of second electrodes 21 is eight, the second electrodes 21 are disposed, for example, on the upper, lower, left, right, upper-right, lower-right, upper-left, and lower-left sides, one on each side.

### 2-4. Pressure Sensitive Resistor

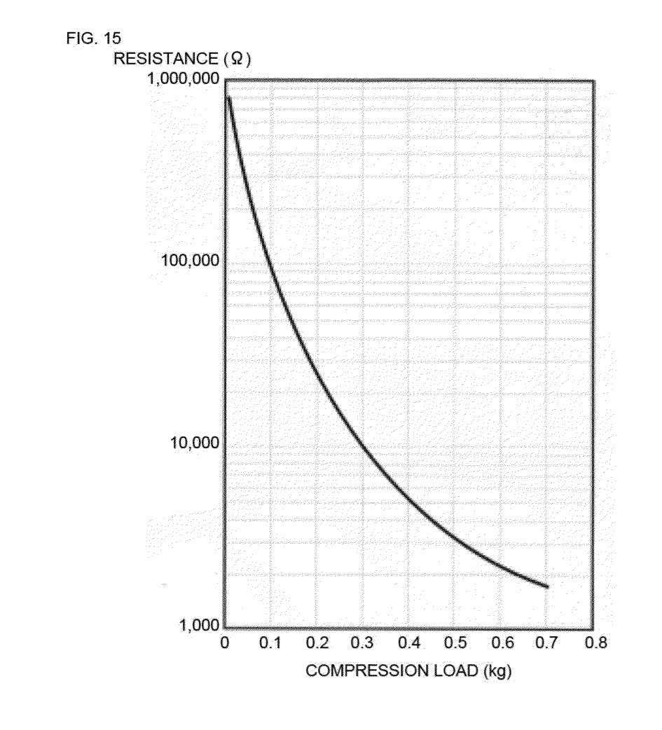

The pressure sensitive resistor 23 is a resistor whose resistance varies with the applied pressure. The pressure sensitive resistor 23 is, for example, an elastomer including a conductive material. An elastomer, which has insulating properties, originally has an extremely high resistance. However, if the elastomer is mixed with a conductive material including conductive fine particles, the resistance of the resulting elastomer decreases according to the applied pressure. Such an elastomer serves as a pressure sensitive sensor. An elastomer including a conductive material is commercially available under the name of a conductive elastomer or elastomer resistor and is available, for example, from Japan Micro System Co., Ltd. FIG. 15 shows an example of pressure-resistance characteristics of an elastomer resistor. In FIG. 15, the resistance logarithmically decreases as the applied pressure linearly increases.

The pressure sensitive unit 15 detects a resistance variation between the first electrode 25 and each second electrode 21 to detect the magnitude of the applied pressure. Accordingly, the pressure sensitive resistor 23 only has to be disposed between the first electrode 25 and the second electrodes 21. Note that the pressure sensitive resistor 23 is preferably disposed in such a manner to surround the first electrode 25 in a section perpendicular to the circumferential direction of the first electrode (a section of FIG. 2(d)). Such a configuration prevents the first electrode 25 from directly contacting the intestinal wall, allowing avoidance of the risk that the first electrode 25 may injure the intestinal wall.

Since the pressure sensitive resistor 23 is sensitive to pressure, it is preferably fixed to the insertion unit 9 without applying an excessive pressure thereto. In the present embodiment, the pressure sensitive resistor 23 is fixed using an adhesive or pressure sensitive adhesive disposed in areas 27 (FIG. 2(a)) between adjacent two second electrodes. By using such a method, the pressure sensitive resistor 23 can be fixed without applying a pressure thereto.

If the pressure sensitive resistor 23 is formed of a material which is relatively easily degraded or contaminated, it is preferred to employ a configuration which allows the pressure sensitive resistor 23 to be replaced. For example, the first electrode 25 and the pressure sensitive resistor 23 covering the first electrode 25 are replaced together. For example, replacement is possible by disposing an electrode in the insertion unit 9 and then bringing this electrode and the first electrode 25 into contact with each other in such a manner to be separable from each other. In another example, it is possible to electrically connect the first electrode 25 and the signal processing unit 5 by disposing a signal line extending from the first electrode 25 along the outside of the insertion unit 9.

### 2-5. Recess on Ridge Line

As shown in FIGS. 2(b) and 2(d), the ridge line of the front end 13 of the insertion unit 9 has a ring-shaped recess in which the second electrodes 21 and the pressure sensitive resistor 23 are disposed. Thus, the second electrodes 21 and the pressure sensitive resistor 23 are stably disposed. A section perpendicular to the circumferential direction, of the recess may be in an L-shape as shown in FIG. 2(d) or in an arc shape as shown in FIG. 3. In the former case, the contact area between the second electrodes 21 and the pressure sensitive resistor 23 is small, and the pressure sensitive resistor may not be properly compressed depending on the direction of a pressure applied to the pressure sensitive resistor. In this case, the pressure may not be measured accurately. In the latter case, the contact area between the second electrodes 21 and the pressure sensitive resistor 23 is large, and the pressure sensitive resistor is properly compressed, regardless of the direction of a pressure applied to the pressure sensitive resistor. Thus, the pressure seems to be measured accurately. As shown in FIG. 4, the recess may have a shape having adjacent three sides of a regular octagon. A recess having such a shape is relatively easily formed. By forming the second electrodes 21 along such a recess, any of a pressure applied to the side surface perpendicularly, a pressure applied to the front end, and a pressure applied diagonally can be properly detected.

Preferably, the recess of the ridge line has a size and shape such that the pressure sensitive resistor 23 extends off at least one of the side and front of the insertion unit 9. While, in FIG. 3, the pressure sensitive resistor 23 extends off both the side and front of the insertion unit 9, it may extend off one of them. Even when the recess is in an L-shape as shown in FIG. 2(d), the pressure sensitive resistor 23 preferably extends off at least one of the side and front of the insertion unit 9. When the pressure sensitive resistor 23 extends off the insertion unit 9 to a greater extent, the pressure sensitive resistor 23 contacts the intestinal wall more easily. Thus, the pressure is detected more easily. Preferably, the amount of extension is about ⅕ to ½ the diameter of the pressure sensitive resistor 23. The reason is that too large an extension amount may destabilize the disposition of the pressure sensitive resistor, whereas too small an extension amount reduces the extension effect.

### 2-6. Pressure Detection Principle

As described above, the pressure sensitive resistor 23 is disposed between the first electrode 25 and the second electrodes 21, and the resistance of the pressure sensitive resistor 23 varies with the magnitude of the applied pressure. Accordingly, by measuring the voltage between the first electrode 25 and each second electrode 21 while passing a constant current through the first electrode 25 and the second electrodes 21, the magnitude of the applied pressure can be detected in the form of a voltage. The voltage obtained is transmitted to the signal processing unit 5 through the signal line. Examples of the method for passing a constant current include use of a constant-current diode.

Next, referring to FIGS. 6 and 7, a pressure-applied position detection principle will be described. FIGS. 6 and 7 correspond to FIG. 2(a), and a pressure-applied position is represented by P. For a distinction, the four second electrodes 21 are given numerals 21A to 21D.

When a pressure is applied to the position P of FIG. 6, the pressure sensitive resistor 23 is significantly compressed around the upper second electrode, 21A, so that the resistance thereof is significantly reduced. Thus, the voltage detected by the upper second electrode, 21A, becomes smaller than those detected by the other second electrodes, 21B, 21C, and 21D. Then the signal processing unit 5 makes a comparison among the voltages detected by the four electrodes and determines that the pressure is being applied to the portion in which the smaller voltage has been detected. In the case of FIG. 6, the signal processing unit 5 determines that the pressure is being applied to the vicinity of the second electrode 21A.

When a pressure is applied to the position P of FIG. 7, the pressure sensitive resistor 23 is significantly compressed between the upper second electrode, 21A, and the left second electrode, 21B. Thus, the voltages detected by the second electrodes 21A and 21B are reduced. Since the position P is slightly closer to the second electrode 21A than to the second electrode 21B, the voltage detected by the second electrode 21A is reduced to a greater extent than that detected by the second electrode 21B. As a result, the signal processing unit 5 determines that the pressure is being applied to the position which lies between the second electrodes 21A and 21B and which is closer to the second electrode 21A than to the second electrode 21B.

### 3. Display of Pressure Information

As described above, the pressure sensitive unit. 15 transmits a signal corresponding to the applied pressure to the signal processing unit 5. The signal processing unit 5 acquires the magnitude and position of the pressure applied to the pressure sensitive unit 15 on the basis of the signals transmitted by the pressure sensitive unit 15. As described above, the pressure sensitive unit 15 can detect the pressure-applied position on the ridge line of the front end 13 of the insertion unit 9. Accordingly, each signal from the pressure sensitive unit 15 includes position information. The signal processing unit 5 refers to this position information and notifies the operator of the pressure-applied position.

Examples of the notification method include output of an alarm signal to give an alarm, display of an alarm on the monitor 7, and display of an alarm on an external display unit which differs from the monitor 7. Examples of the alarm display method include a change in the color or luminance of the screen and display of characters or symbols on the screen. The notification method may be transmission of a notification only when a high pressure exceeding a reference value is applied, or real-time display of the detected pressure using a value or color, regardless of the magnitude of the applied pressure.

One example of the notification method is shown in FIG. 8. That is, a display 31 based on the acquired magnitude of the applied pressure is made in a position which is located adjacent to an image 29 captured by the endoscope 3 and which corresponds to the acquired pressure-applied position. For example, if the front end 13 of the insertion unit 9 is contacting the lower side of the intestine and thus a pressure is being applied to the lower side of the pressure sensitive unit 15, the signal processing unit 5 determines that the pressure-applied position lies on the “lower side.” Accordingly, a display 31 indicating the acquired applied pressure is made on the “lower side” of the image 29 (see FIG. 8). The display 31 is, for example, a color corresponding to the magnitude of the applied pressure but may be a pattern, characters, symbols, or the like. For another example, if the signal processing unit 5 determines that the pressure-applied position lies on the “right side,” a display 31 indicating the acquired applied pressure is made on the “right side” of the image 29. Alternatively, if the signal processing unit 5 determines that the pressure-applied position lies on the “right side,” a display 31 indicating the acquired applied pressure is made on the “left side” of the image 29. The reason is as follows: when a pressure is being applied to the right side of the pressure sensitive unit 15, the operator needs to move the front end 13 of the insertion unit 9 to the left; and therefore making a display in the moving direction may help the operator understand the moving direction intuitively. The display positions adjacent to the image 29 may be four positions consisting of upper, lower, left, and right positions, eight positions including diagonal directions, or more positions. For example, the number of display positions is the same as the number of second electrodes.

When the operator looks at the display 31, he or she recognizes that the front end 13 of the insertion unit 9 is contacting the lower side of the intestine and then operates the operation unit 11 to move the front end 13 upward. Thus, the operator can avoid intestinal perforation. Further, the supervisor of the operator can confirm the pressure applied to the intestine and thus early determine an intestinal perforation risk.

Another embodiment of the method for notifying the operator of a pressure is to make a display on an external display unit 33 which differs from the monitor 7, as shown in FIG. 9. For example, the external display unit 33 has one display part 35 in each of upper, lower, left, and right areas thereof, and a display based on the acquired magnitude of the applied pressure is made on a display part. 35 corresponding to the acquired pressure-applied position. For example, each display part. 35 may be provided with LEDs of multiple colors and notify the operator of the magnitude of the applied pressure by emitting light of a color corresponding to the magnitude of the applied pressure.

For example, the external display unit 33 may be mounted on the monitor 7. By using such an external display unit 33, it is possible to notify the operator of a pressure without having to make a change to the existing monitor.

If the magnitude of the pressure is displayed using a color, examples of the color are as follows.

TABLE 1

| Magnitude of Applied Pressure | Color |
|---|---|
| <1 kg/cm² | Black |
| 1 kg/cm²≦ and 2 kg/cm² < | Blue |
| 2 kg/cm²≦ and 2.5 kg/cm² < | Green |
| 2.5 kg/cm²≦ and 3 kg/cm² < | Yellow |
| 3 kg/cm²≦ and 3.5 kg/cm² < | Orange |
| 3.5 kg/cm²≦ and 4 kg/cm² < | Red |
| 4 kg/cm²≦ | White |

### 4. Adapter Pressure Sensor

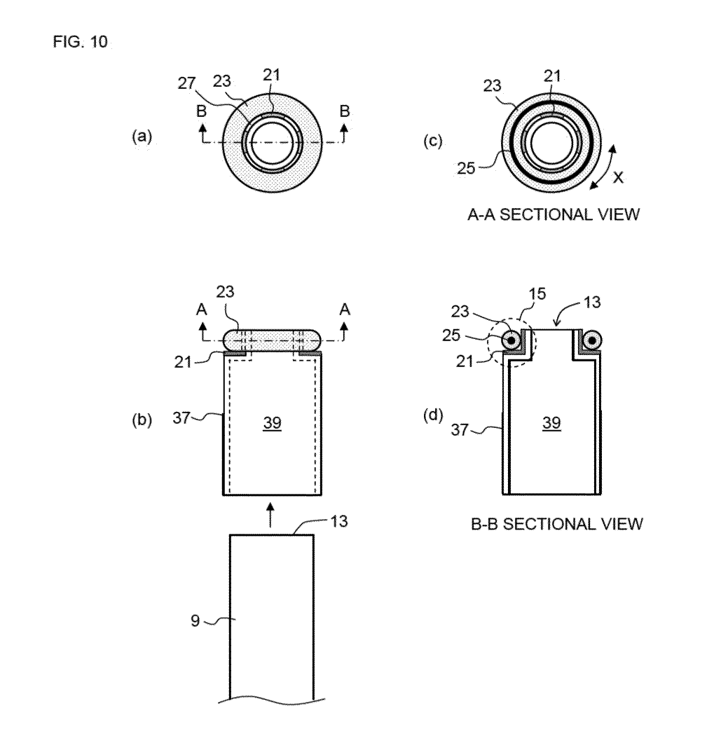

While the case where the pressure sensitive unit 15 is mounted on the front end 13 of the insertion unit 9 has been described above, the pressure sensitive unit 15 may be mounted on an adapter 37 which is detachable from the front end 13 of the insertion unit 9, as shown in FIGS. 10(a) and 10(d). FIGS. 10(a) to 10(d) correspond to FIGS. 2(a) to 2(d), respectively. The insertion unit 9 of the endoscope 3 is inserted into internal space 39 of the adapter 37. Use of such an adapter pressure sensor has an advantage that the pressure sensitive unit 15 can be easily replaced. Another advantage is that such an adapter pressure sensor can also be mounted on the insertion units of traditional endoscopes. Use of the adapter 37 and the external display unit. 33 allows the present invention to be introduced to the existing endoscopes at hospitals and the like. The first electrode 25 and the second electrode 21 are connected electrically to the signal processing unit 5 through signal lines (not shown) in the adapter 37. These signal lines can be connected to the signal processing unit 5 along the insertion unit 9.

Such a configuration, in which the pressure sensitive unit 15 is disposed on the adapter 37, can be said to be a pressure sensor which can be used in any application. This pressure sensor can be used in endoscopes, as well as in any applications where the position and magnitude of the applied pressure need to be detected. The adapter 37 corresponds to the “support unit.” in the claims.

5. Configuration where Positions of First and Second Electrodes are Inverted

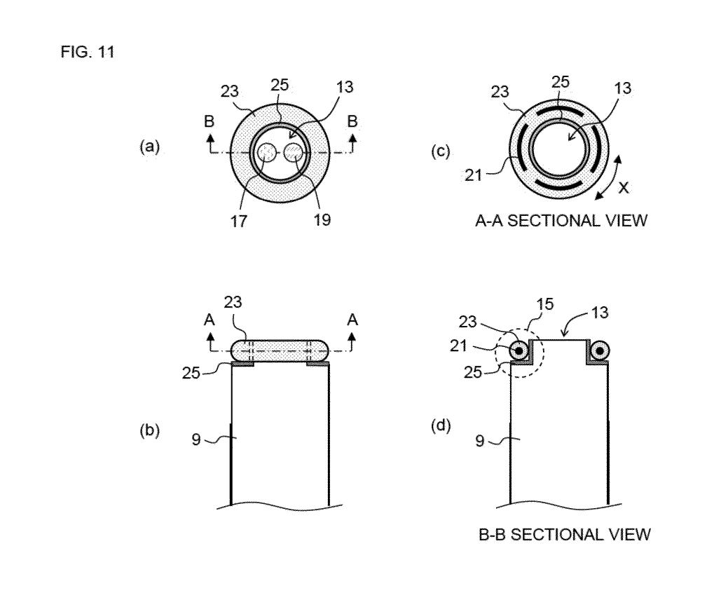

There has been described the embodiment above where the first electrode 25 is disposed inside the pressure sensitive resistor 23 and where the second electrodes 21 are disposed adjacent to the insertion unit 9. Alternatively, as shown in FIGS. 11(a) to 11(d), there may be employed an embodiment where a ring-shaped first electrode 25 is disposed adjacent to the insertion unit 9 and where multiple second electrodes 21 are disposed as opposed to the first electrode 25. In this embodiment, a pressure sensitive resistor 23 is formed in such a manner to surround the second electrodes 21 in a section perpendicular to the circumferential direction of the second electrodes 21.

Such a configuration also allows detection of the magnitude and position of the pressure applied to the pressure sensitive unit 15 on a principle similar to that of the above embodiment. The description of the above embodiment also applies to the present embodiment without departing from the scope thereof.

### EXAMPLE

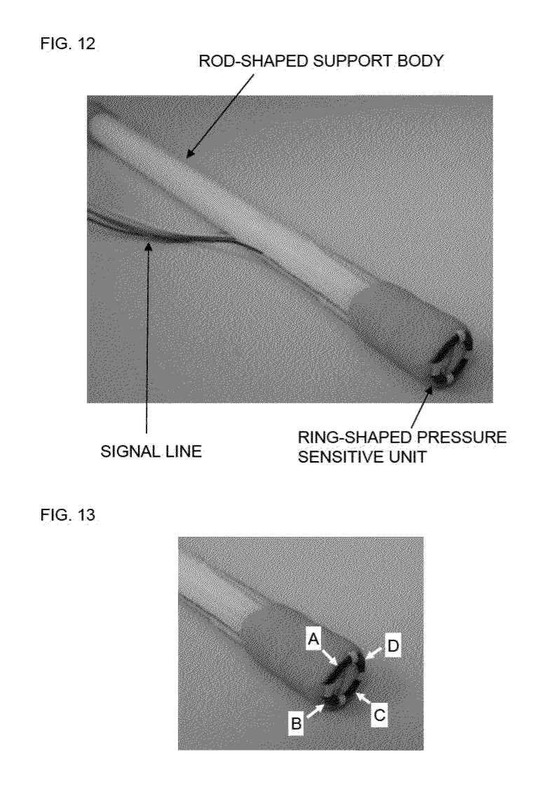

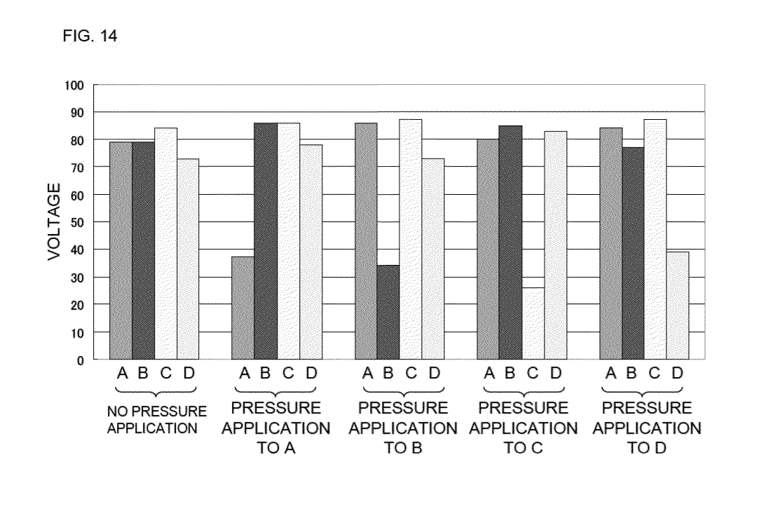

Next, an Example of the present invention will be described. FIG. 12 shows an endoscope model used in this Example. A ring-shaped pressure sensitive unit is disposed on the ridge line of the front end of a rod-shaped support body. This pressure sensitive unit is formed by covering a ring-shaped first electrode with a pressure sensitive resistor (elastomer resistor) and then spacing four second electrodes uniformly in the circumferential direction of the first electrode. Pressures were applied to positions shown by arrows A to D in FIG. 13 by sequentially pushing these positions using a finger while passing a constant current through the first and second electrodes, and the then voltage between the first electrode and each second electrode was measured. The results are shown in FIG. 14.

As shown in FIG. 14, when the pressure was applied to each of the positions shown by arrows A to D, the voltage of the pressure-applied position was reduced. Note that the vertical axis of FIG. 14 represents a percentage with respect to a reference voltage 100. FIG. 14 demonstrates that the magnitude and position of the applied pressure can be detected.

### DESCRIPTION OF NUMERALS

1: endoscope device, 3: endoscope, 5: signal processing unit, 7: monitor, 9: insertion unit, 13: front end, 11: operation unit, 15: pressure sensitive unit, 17: projector, 19: image capturing unit, 25: first electrode, 21, 21A to 21D: second electrode, 23: pressure sensitive resistor, 27: area between adjacent two second electrodes, 29: image, 31: display, 33: external display unit, 35: display part, 37: adapter, 39: internal space

1. A pressure sensor comprising:
a support unit; and
a pressure sensitive unit disposed on a ridge line of a front end of the support unit and configured to output a signal corresponding to an applied pressure, wherein
the pressure sensitive unit comprises:
a first electrode disposed in a ring shape; and
a plurality of second electrodes opposed to the first electrode, and
the second electrodes are spaced in a circumferential direction of the first electrode.

2. The pressure sensor of claim 1, wherein
the pressure sensitive unit further comprises a pressure sensitive resistor disposed between the first and second electrodes, a resistance of the pressure sensitive resistor varying with an applied pressure.

3. The pressure sensor of claim 2, wherein
the pressure sensitive resistor comprises an elastomer comprising a conductive material.

4. The pressure sensor of claim 3, wherein
the pressure sensitive resistor surrounds the first or second electrode in a section perpendicular to a circumferential direction of the first or second electrode.

5. The pressure sensor of claim 2, wherein
the support unit has a ring-shaped recess on the ridge line, and
at least one of the first and second electrodes, and the pressure sensitive resistor are disposed in the recess.

6. The pressure sensor of claim 5, wherein
a section of the pressure sensitive resistor is circular, the section being perpendicular to a circumferential direction of the ridge line, and
a section of the recess is in an arc shape, the section being perpendicular to the circumferential direction of the ridge line.

7. The pressure sensor of claim 2, wherein
the pressure sensitive resistor extends off at least one of a side and a front of the support unit.

8. The pressure sensor of claim 2, wherein
the second electrodes are disposed on the support unit, and
the pressure sensitive resistor is fixed to the support unit using an adhesive or pressure sensitive adhesive disposed between adjacent two second electrodes.

9. The pressure sensor of claim 1, wherein
the second electrodes comprise four or more second electrodes uniformly spaced in the circumferential direction of the first electrode.

10. An endoscope comprising:
the pressure sensor of claim 1; and
an insertion unit configured to be inserted into a body, wherein
the support unit constitutes a front end portion of the insertion unit.

11. An endoscope comprising:
the pressure sensor of claim 1; and
an insertion unit configured to be inserted into a body, wherein
the support unit is an adapter configured to detachably fix the pressure sensitive unit to the insertion unit.

12. An endoscope device:
the endoscope of claim 10;
a signal processing unit configured to acquire a magnitude of a pressure applied to the pressure sensitive unit and a position to which the pressure has been applied, based on a signal from the pressure sensitive unit; and
a monitor configured to display an image captured by the endoscope.

13. The endoscope device of claim 12, wherein
the signal processing unit makes a display based on the magnitude of the applied pressure in a position which is located adjacent to the image and which corresponds to the pressure-applied position.

14. The endoscope device of claim 12, further comprising
an external display unit which differs from the monitor, wherein
the signal processing unit makes a display based on the magnitude of the applied pressure in a position on the external display unit, the position corresponding to the pressure-applied position.

15. The endoscope device of claim 13, wherein
the display is a color corresponding to the magnitude of the applied pressure.
